(19) United States
(12) Patent Application Publication
(10) Pub. No.: US 2014/0017390 A1
(43) Pub. Date: Jan. 16, 2014

(54) Tableting Aid

(71) Applicants: Reinhard Vollmer, Windesheim (DE); Tobias Goetz, Rosenberg (DE)

(72) Inventors: Reinhard Vollmer, Windesheim (DE); Tobias Goetz, Rosenberg (DE)

(21) Appl. No.: 13/982,739

(22) PCT Filed: Feb. 1, 2012
(86) PCT No.: PCT/EP2012/000441

§ 371 Date: Sep. 20, 2013

(30) Foreign Application Priority Data
Feb. 4, 2011 (DE) 10 2011 010 437.2

(51) Int. Cl. A23L 1/30; A61K 9/20

(52) U.S. Cl. CPC A23L 1/30; A61K 9/2054; A61K 9/2009; A61K 9/2013
USPC 426/658

(57) ABSTRACT

The invention relates to a directly compressible tableting excipient for use in foodstuffs (dietary supplement), containing:

78-97% of a binding agent;

0.1-5% of an emulsifier;

0.1-5% of a vegetable or animal fat.

In the field of pharmaceutical technology, the tablet is the most common form of compressing active agents. Consequently, there is a wide knowledge in pharmacy and a lot of experience in the production of tablets. In addition, there is a wide range of inactive ingredients for the manufacture of tablets, granulates and other solid forms.

The inactive ingredients for the production of medicinal products are defined and described in general in official dispensatories (pharmacopoeia). There are binding agents, mold release agents, lubricants, disintegrants, solubilizing agents, and many others.

In addition to the medicinal products, so-called dietary supplements have become known in recent years. The dietary supplements are intended to offset deficits in essential nutrients that are not or not sufficiently provided to the human body under certain circumstances.

Dietary supplements include vitamin and mineral preparations, preparations of amino acids, enzymes, trace elements and various plant extracts.

Dietary supplements are subject in most countries to foodstuff legislation. Accordingly, the list of the permitted inactive ingredients for pressing tablets is very limited. These inactive ingredients are mention for example in the Directive 96/77/EC of the Commission. However, some additives are not listed there, thus not allowed for dietary supplements and foods, but still for pharmaceutical applications.

It would be extremely desirable to have a directly compressible tableting excipient which offers the advantages of such tableting excipients that are successfully used in the field of pharmaceutical technology, and can be approved at the same time for use in foodstuffs and dietary supplements. The desired properties are substantially the following:
High tablet hardness
Good lubricating properties
Uncritical concerning long mixing durations

Known tableting excipients usually contain individual substances, which cause one of these properties. Previously, these individual substances needed to be added in a dosed manner to the active ingredient, often in a specific sequence or in certain time intervals.

Although there are individual substances which have the required properties, and which also satisfy the requirements of food legislation, they are not suitable to be combined in a simple way with all other necessary individual substances. For example, magnesium stearate is an excellent lubricant, and also approved in the food industry. However, it cannot adequately emulsify at room temperature, but would need to be heated first over the melting point and added in liquid form, which is energetically costly and would entail other problems.

The invention is thus based on the object of providing a directly compressible tableting excipient and a method for its production, by means of which the same or similar characteristics can be obtained as in a pharmaceutical tableting excipient, but at the same time meets the requirements of foodstuff legislation. The partial object is very important to find tableting excipients which can be subjected to co-processing, in which the starting materials are provided at room temperature or slightly above, at least without the necessity of heating to three digit temperature levels in order to obtain a final compound, to which only the active agent of the tablet to be produced needs to be added.

This object is achieved by the features of the independent claims.

The tableting excipient in accordance with the invention therefore contains as the necessary components a binding agent such as microcrystalline cellulose, an emulsifier such as lecithin, as well as a vegetable or animal fat as a lubricant. Such a tableting excipient offers the following advantages: it meets the requirements of the foodstuff legislation, it contains all necessary individual substances that have the desired properties necessary for tableting, co-processing can be performed at room temperature or at an only slightly elevated temperature, and therefore virtually without any energy input, so that a complete tableting excipient is obtained which only needs to be mixed with the active agent of the tablet.

A tableting excipient according to the invention may have the following composition:
78-97% of a binding agent, e.g. microcrystalline cellulose
1 to 10% of a glidant, e.g. colloidal silicate
0.5 to 5% of a disintegrant, e.g. cross-linked sodium carboxymethyl cellulose
0.1 to 5% of a lubricant, e.g. saturated vegetable oil
0.1 to 5% of an emulsifier, e.g. lecithin

The use of sodium stearyl fumarate is known as a lubricant from WO 2009/112287 A1. Due to its chemical structure, sodium stearyl fumarate not only acts as a lubricant but also as an emulsifier.

This is not allowed in the food industry, however. With the combination of lecithin/vegetable fat in accordance with the invention, a compound was found which acts as a lubricant and as an emulsifying agent, which is also approved under the foodstuff legislation.

The excipients as mentioned above are linked to each other in the manner described below in such a way that a compound is obtained for direct pressing of tablets and granulates.

The compound according to the invention offers the following advantages compared to the physical mixture of individual components:
1. By fixing the lubricating substances—hydrogenated vegetable fat and lecithin—to microcrystalline cellulose as the binding agent, the binding particles are no longer lubricated during the mixing process. This results in significantly higher tablet hardness than with the physical mixture.
2. The concentration of the fixed lubricating agents is adequate to sufficiently lubricate the matrix of the tablet press during the pressing process. A further addition of lubricants such as magnesium stearate can be omitted.
3. Thanks to the uniform distribution of the fixed lubricant, the tablet ejection forces are reduced.
4. Longer mixing times have no negative effect on the tablet hardness. Poorly miscible substances can be mixed for a sufficiently long time.
5. The main advantage of the invention is that the active ingredient only needs to be mixed with the tableting excipient. Compression can take place immediately thereafter. No other additives are required.

The production of the compound as described above from the aforementioned components preferably occurs by wet granulation according to all known methods such as mixing granulation, perforated-disc granulation, fluidized bed granulation, extrusion or Shugi granulation.

Granulation in the fluidized bed can be performed as follows for example:
I. Silicified microcrystalline cellulose and cross-linked sodium carboxymethyl cellulose are supplied to the fluidized-bed dryer. The mixture is heated to 30 to 35° C. At the same time, 10 parts of coconut oil, 10 parts of lecithin and 100 parts of water are emulsified with high shear forces. The resulting emulsion is slowly sprayed through a top spray nozzle onto the supplied cellulose. The material is dried in an air stream to a moisture content of 3.0 to 3.5%.
II. Microcrystalline cellulose and cross-linked sodium carboxymethyl cellulose are supplied to a fluidized-bed dryer. At the same time, 10 parts of coconut oil, 10 parts of lecithin, 2 parts of colloidal silica and 98 parts of water are emulsified with high shear forces. The resulting emulsion is slowly sprayed through a top spray nozzle onto the supplied cellulose. The material is dried in an air stream to a moisture content of 3.0 to 3.5%. In both cases a free-flowing, directly compressible powder is obtained.

### Tableting Trials

The compound produced cannot be compared directly with the physical mixture of the individual components, as the vegetable oil cannot mix in the pure state with the microcrystalline cellulose. That is why classical formulations were compared by using pure microcrystalline cellulose as the binding agent and magnesium stearate as the lubricant.

Example of asorbic acid tablet:

| | Ratio of active | Classical |
|---|---|---|
| Ingredients | ingredient to excipient | mixture |
| Ascorbic acid | 50% | 50% |
| Microcrystalline cellulose | | 45% |
| Silicon dioxide | | 2% |
| Sodium carboxyl methyl | | 2% |
| cellulose, cross-linked | | |
| Magnesium stearate | | 1% |
| Tableting excipient | 50% | |

| | Tableting | Classical |
|---|---|---|
| Test parameters | excipient | mixture |
| Pressing force (kN) | 7.0-10.0 | 7.0-10.0 |
| Ejection force (N) | 90-95 | 110-130 |
| Tablet hardness (N) | 120-170 | 90-110 |
| Disintegration time (sec) | 30-40 | 30-40 |

Example of plant extract tablet:

| | Ratio of active | Classical |
|---|---|---|
| Ingredients | ingredient to excipient | mixture |
| Plant extract | 50% | 50% |
| Microcrystalline cellulose | | 45% |
| Silicon dioxide | | 2% |

| | | |
|---|---|---|
| Sodium carboxyl methyl | | 2% |
| cellulose, cross-linked | | |
| Magnesium stearate | | 1% |
| Tableting excipient | 50% | |

| | Tableting | Classical |
|---|---|---|
| Test parameters | excipient | mixture |
| Pressing force (kN) | 7.0-10.0 | 7.0-10.0 |
| Ejection force (N) | 50-70 | 100-120 |
| Tablet hardness (N) | 110-140 | 100-115 |
| Disintegration time (sec) | 60-80 | 70-110 |

The tableting excipient in accordance with the invention can be compressed with many active ingredients commonly used in the field of dietary supplements. These are vitamins, pure or in mixtures, minerals, pure or in mixtures, plant extracts, enzymes, and amino acids and other substances such as glucosamine, chondroitin, methylsulfonyl sulphate and more.

The directly compressible tableting excipient may also be prepared by using the following components:
a) Microcrystalline cellulose, powdered cellulose, various cellulose derivatives (hydroxymethyl propyl cellulose), polyols (sorbitol, mannitol) of sugars (sucrose, glucose, fructose), in the respective dosage of 78 to 97%, as binding agents.
Silicon dioxide of various types, aluminum silicates, in the respective dosage of 1 to 10% as glidants.
c) Cross-linked sodium carboxymethyl cellulose, polyvinyl pyrrolidone, starch, in the respective dosage dosage of 0.5 to 5%, as disintegrants.
d) Lecithin or other phosphatides, monoglycerides or diglycerides, in the respective dosage of 0.1 to 5%, as emulsifiers.
e) Vegetable or animal fats or oils, in the respective dosage of between 0.1 and 5%, as lubricants.

1-5. (canceled)

6. A directly compressible tableting excipient for use in foods, the directly compressible tableting excipient comprising:
78-97% of a microcrystalline cellulose or a silicified microcrystalline cellulose;
a lecithin or phosphatide or monoglyceride or diglyceride as an emulsifier;
0.1-5% of a saturated vegetable fat as a lubricant;
a colloidal silicon dioxide as a glidant;
sodium carboxymethyl cellulose as a disintegrant.

7. A method for producing the directly compressible tableting excipient of claim 6, the method comprising:
performing wet granulation including mixing granulation or perforated-disc granulation or fluidized-bed granulation or extrusion or Shugi granulation or spray drying.
